(19) United States
(12) Patent Application Publication
(10) Pub. No.: US 2025/0046450 A1
(43) Pub. Date: Feb. 6, 2025

(54) SYSTEM AND METHOD FOR PROVIDING ENHANCED MEDICAL SYMPTOM CHECKER

(71) Applicant: JIO PLATFORMS LIMITED, Ahmedabad (IN)

(72) Inventors: Chetan B. BHATT, Mumbai (IN); Amrita Virendra VERMA, Mumbai (IN); Ritesh SHAH, Hyderabad (IN); Kalyan TADEPALLI, Mumbai (IN); Neeraj YOGENDRA, Hyderabad (IN); Vamshi CHITTALA, Hyderabad (IN); Hareesh KUMAR, Hosur (IN); Shailesh KUMAR, Hyderabad (IN)

(73) Assignee: JIO PLATFORMS LIMITED, Ahmedabad (IN)

(21) Appl. No.: 18/713,962

(22) PCT Filed: Nov. 25, 2022
(86) PCT No.: PCT/IB2022/061429

§ 371 Date: May 28, 2024

(30) Foreign Application Priority Data
Nov. 29, 2021 (IN) 202121055282

(51) Int. Cl. G16H 50/20; G16H 10/60

(52) U.S. Cl. CPC G16H 50/20; G16H 10/60

(57) ABSTRACT

The present invention provides solution to the above-mentioned problem in the art by providing a system and a method for efficiently identifying from a medical domain a plurality of entities that may be designed and engineered for a diagnostic reasoning system or symptom checker. The identified plurality of entities forms nodes of a knowledge graph and may be interconnected to each other with edges representing finely curated weights. The curated weights may form a dynamic repository that can constantly evolves to best reflect the latest medical knowledge. Apart from diseases and symptoms, the system is also capable of extending and scaling to other dimensions of medical knowledge. The knowledge graph thus developed is fundamental to the entire diagnostic system and is used by various components of the diagnostic engine to power the AI-based reasoning.

### FIELD OF INVENTION

The embodiments of the present disclosure generally relate to the field of medical engineering. More particularly, the present disclosure relates generally to system and method providing a built state-based design of an artificial intelligence (AI) engine and an expert curated medical knowledge pre-clinical diagnosis of diseases.

### BACKGROUND OF THE INVENTION

The following description of related art is intended to provide background information pertaining to the field of the disclosure. This section may include certain aspects of the art that may be related to various features of the present disclosure. However, it should be appreciated that this section be used only to enhance the understanding of the reader with respect to the present disclosure, and not as admissions of prior art.

Disease diagnosis is a complex problem due to typical similarities and dissimilarities among diseases symptoms (e.g. fever, headache), symptom attributes (e.g. degree, duration), test results, medical history, lifestyle choice, weather and location priors and so on. Typically, a doctor can take many aspects into account to make an accurate diagnosis. Building an artificial intelligence (AI) based Symptom Checker to mimic the real doctor's diagnosis process requires us to have both the knowledge base of all medical knowledge that a doctor has and also the reasoning capabilities to navigate through this knowledge base in the context of each patient. In this patent, we describe how the medical knowledge especially as it pertains to disease, symptoms and symptom attributes is represented to help the diagnosis process.

Knowledge engineering is a fundamental part of any expert-based AI system. It deals with designing the correct representation and schema for the expert knowledge in a way that is well-suited to the use-case and the system architecture. Online symptom checkers are health tools that help patients and doctors diagnose the most likely disease(s) for any combination of symptoms and their attributes. In the healthcare domain, a symptom-based triage is known to be a critical and complex use-case due to various factors such as the impact and subjectivity of diagnoses, knowledge engineering of the vast medical information and the inferencing required thereof. Online symptom checkers find significant applications in providing quick diagnoses to patients who do not have easy access to health facilities. Such tools can also be referred to by medical students to learn differential diagnosis. The existing online symptom-checkers or diagnostics-as-a-service products make use of medical ontologies and web-data coupled with knowledge-based or empirical based approaches to achieve accurate disease predictions. They are available either as products or online web applications. Few employ an interactive methodology in the form of question-answers in a chatbot-like setting whereas some predict diseases based only on initial symptoms entered by the user. The key factors that interactive symptom-checkers are typically evaluated upon are as follows.
Accuracy of the final differential diagnosis
Coverage of diseases and symptoms
Correctness/Relevance of questions asked
Order in which the questions are asked
Number of questions that need to be answered

Existing online symptom checkers employ various automatic methods to elicit the required medical data from the web. Often due to inconsistent or limited structured and standardized datasets on the web, this potentially leads to an inaccurate and incomplete knowledge base at the outset. Furthermore, such data when used to train various machine learning models tend to propagate these errors eventually resulting in less accurate diagnosis.

There is therefore a need in the art to provide for a-built state-based design of an Al-engine and an expert curated medical knowledge to alleviate issues in prior art, allow for scaling and modelling of additional medical entities and facilitate gradual improvements in the parameterised algorithms.

### OBJECTS OF THE PRESENT DISCLOSURE

Some of the objects of the present disclosure, which at least one embodiment herein satisfies are as listed herein below.

It is an object of the present disclosure to provide a system and a method that facilitates a schema design comprising of primary entities related to diagnosis in the form of diseases and symptoms.

It is an object of the present disclosure to provide a knowledge graph to represent the interconnections between the primary entity nodes in the form of expert curated weighted edges

It is an object of the present disclosure to provide a system and a method that enables design of various types of weights in order to improvise the differential diagnosis.

It is an object of the present disclosure to provide a system and a method that allows for modelling dependencies between symptoms, symptom attributes and symptom-attribute values further providing a possibility to ask deep conditional questions and improve the specificity of diagnosis.

It is an object of the present disclosure to provide a system and a method that includes more than 20,000 highly curated units of knowledge.

It is an object of the present disclosure to provide a system and a method that facilitates coverage of more than a 1000 most common diseases and more than a 1000 most common symptoms and their attributes.

It is an object of the present disclosure to provide a system and a method that facilitates complex graph with more than a billion possible connections.

It is an object of the present disclosure to provide a schema design that allows for factoring in other dimensions of medical knowledge, called contexts, beyond diseases, symptoms, and symptom attributes whose combination with these primary entities fine-tunes and adapts the diagnosis by such parameter and further increases the accuracy of diagnosis prediction. Context is looked at two ways by the system: group specific and disease specific.

It is an object of the present disclosure to provide a system and a method that is not limited to demographic data, climate conditions, geolocation, lifestyle parameters, medical history, surgical history, drug history, laboratory and imaging tests, occupation, addiction(s) or habit(s) etc.

It is an object of the present disclosure to provide a system and a method that is a dynamically evolving entity and is capable of being scaled exponentially.

It is an object of the present disclosure to provide a system and a method that enables visual representations of the encoded medical knowledge in a way that will be casily and accurately comprehensible to the patient is a plurality of representations such as textual, clickable humanoid representations, Color-coded scales, Carousel of Images, Static images and GIFs (Graphics Interchange Format) and the like.

### SUMMARY

This section is provided to introduce certain objects and aspects of the present disclosure in a simplified form that are further described below in the detailed description. This summary is not intended to identify the key features or the scope of the claimed subject matter.

In an aspect, the present disclosure provides for a system for providing enhanced medical symptom checker. The system may include one or more processors operatively coupled to a plurality of first computing devices, the one or more processors coupled with a memory that stores instructions which when executed by the one or more processors causes the system to receive an input query from one or more users associated with the plurality of first computing devices, the input query pertaining to one or more symptoms associated with a disease. The system may be further configured to extract a first set of attributes from the received input query, the first set of attributes pertaining to the one or more symptoms of the disease. The system may be further configured to extract a second set of attributes from the received input query, the second set of attributes pertaining to demographic conditions and location. Based on the extracted first and second attributes, the system may be configured to map the one or more symptoms with a set of parameters determined by searching a medical knowledgebase for similarity of the set of parameters in the one or more symptoms. The medical knowledgebase may include static medical resources, medical databases, online medical resources, and peer reviewed journals for a target disease. The system may be further configured to diagnose the disease based on the mapped one or more symptoms with the set of parameters associated with the target disease.

In an embodiment, the system may be further configured to collect a plurality of points of data chosen by a plurality of medical experts to gather, refine and enrich the medical knowledgebase.

In an embodiment, the system may be further coupled to an interface unit configured to display a plurality of information associated with the diagnosed disease.

In an embodiment, the system may be further configured to perform a feedback analysis executed by the plurality of medical experts. The feedback analysis may be based on a plurality of triages performed by the plurality of medical experts mimicking a plurality of clinical scenarios, and each triage may include the target disease, age, gender, one or more symptoms and context applicable to the plurality of clinical scenarios.

In an embodiment, the system may be further configured to generate one or more derived symptoms based on one or more symptom questions and an initial set of complaints provided by a user.

In an embodiment, the one or more symptoms may include the set of initial symptoms and the one or more derived symptoms.

In an embodiment, the system may be further configured to find out a plurality of abnormalities or medically incorrect responses generated by the system.

In an embodiment, the system may be further configured to improve and fix a knowledge graph comprising one or more knowledge features associated with the medical knowledgebase, update the one or more knowledge features in the knowledge graph, and add a new feature to the knowledge graph.

In an embodiment, the knowledge graph may be coupled with appropriate visual representations of a medical knowledge that is easily and accurately comprehensible to the user.

In an embodiment, the system may be further configured to allow for factoring in a plurality of dimensions of medical knowledge whose dynamic nature works to augment the accuracy of diagnosis prediction.

In an embodiment, the system may be further coupled to a user interface configured with an intuitive dialogue between the user and the system.

In an embodiment, the user interface may be configured with one or more clickable interactive humanoid representations. The user may choose one or more clickable interactive humanoid representations that represent the part that the user has the pain at.

In an aspect, the present disclosure provides for a user equipment (UE) for providing enhanced medical symptom checker. The UE may include a processor and a receiver operatively coupled to a plurality of first computing devices, the processor coupled with a memory that stores instructions which when executed by the processor causes the UE to receive an input query from one or more users associated with the plurality of first computing devices, the input query pertaining to one or more symptoms associated with a disease. The UE may be further configured to extract a first set of attributes from the received input query, the first set of attributes pertaining to the one or more symptoms of the disease. The UE may be further configured to extract a second set of attributes from the received input query, the second set of attributes pertaining to demographic conditions and location. Based on the extracted first and second attributes, the UE may be configured to map the one or more symptoms with a set of parameters determined by searching a medical knowledgebase for similarity of the set of parameters in the one or more symptoms. The medical knowledgebase may include static medical resources, medical databases, online medical resources, and peer reviewed journals for a target disease. The UE may be further configured to diagnose the disease based on the mapped one or more symptoms with the set of parameters associated with the target disease.

In an aspect, the present disclosure provides for a method for providing enhanced medical symptom checker. The method may include the step of receiving, by one or more processors, an input query from one or more users associated with the plurality of first computing devices, the input query pertaining to one or more symptoms associated with a disease. The one or more processors may be operatively coupled to a plurality of first computing devices and may be coupled with a memory that stores instructions which may be executed by the one or more processors. The method may further include the steps of extracting, by the one or more processors, a first set of attributes from the received input query, the first set of attributes pertaining to the one or more symptoms of the disease and extracting, by the one or more processors, a second set of attributes from the received input query, the second set of attributes pertaining to demographic conditions and location. Based on the extracted first and second attributes, the method may further include the step of mapping, by the one or more processors, the one or more symptoms with a set of parameters determined by searching a medical knowledgebase for similarity of the set of parameters in the one or more symptoms. The medical knowledgebase may include static medical resources, medical databases, online medical resources, and peer reviewed journals for a target disease. Furthermore, the method may include the step of diagnosing, by the one or more processors, the disease based on the mapped one or more symptoms with the set of parameters associated with the target disease.

### BRIEF DESCRIPTION OF DRAWINGS

The accompanying drawings, which are incorporated herein, and constitute a part of this invention, illustrate exemplary embodiments of the disclosed methods and systems in which like reference numerals refer to the same parts throughout the different drawings. Components in the drawings are not necessarily to scale, emphasis instead being placed upon clearly illustrating the principles of the present invention. Some drawings may indicate the components using block diagrams and may not represent the internal circuitry of each component. It will be appreciated by those skilled in the art that invention of such drawings includes the invention of electrical components, electronic components or circuitry commonly used to implement such components.

FIG. 1 illustrates an exemplary network architecture (100) in which or with which the system of the present disclosure can be implemented, in accordance with an embodiment of the present disclosure.

FIG. 2A illustrates an exemplary representation (200) of system (110), in accordance with an embodiment of the present disclosure.

FIG. 2B illustrates an exemplary block diagram representation of a user equipment (UE) for recommending region specific personalized news, in accordance with an embodiment of the present disclosure.

FIG. 3 illustrates an exemplary block diagram (300) representation of the proposes system in accordance with an embodiment of the present disclosure.

FIGS. 4A-4D illustrates exemplary flow diagram representation (400) of the proposed method, in accordance with an embodiment of the present disclosure.

FIGS. 5A-5C illustrate exemplary representations of flow diagrams associated with a medical knowledgebase, in accordance with an embodiment of the present disclosure.

FIGS. 6A-6C illustrate exemplary representations of flow diagrams associated with disease-Symptom view, in accordance with an embodiment of the present disclosure.

FIGS. 7A-7B illustrate exemplary representations of tree diagrams associated Diagnostics-as-a-service, in accordance with an embodiment of the present disclosure.

FIG. 8 illustrates an exemplary computer system in which or with which embodiments of the present invention can be utilized in accordance with embodiments of the present disclosure.

The foregoing shall be more apparent from the following more detailed description of the invention.

### BRIEF DESCRIPTION OF INVENTION

In the following description, for the purposes of explanation, various specific details are set forth in order to provide a thorough understanding of embodiments of the present disclosure. It will be apparent, however, that embodiments of the present disclosure may be practiced without these specific details. Several features described hereafter can each be used independently of one another or with any combination of other features. An individual feature may not address all of the problems discussed above or might address only some of the problems discussed above. Some of the problems discussed above might not be fully addressed by any of the features described herein.

The present invention provides solution to the above-mentioned problem in the art by providing a system and a method for efficiently identifying from a medical domain a plurality of entities that may be designed and engineered for a diagnostic reasoning system or symptom checker. The identified plurality of entities form nodes of a knowledge graph and may be interconnected to each other with edges representing finely curated weights. The curated weights may form a dynamic repository that can constantly evolve to best reflect the latest medical knowledge, statistical experience of the country or region it is applied, and the additional qualifiers based on the context. Apart from diseases and symptoms, the system is also capable of extending and scaling to other dimensions of medical knowledge namely, disease priors pertaining to demography, climate conditions, geolocation and the like and risk factors pertaining to lifestyle, medical history, family history and the like, laboratory and imaging tests, body organs, body systems and the like. The knowledge graph thus developed is fundamental to a comprehensive diagnostic system and is used differently by various components of the diagnostic engine to power the AI-based reasoning.

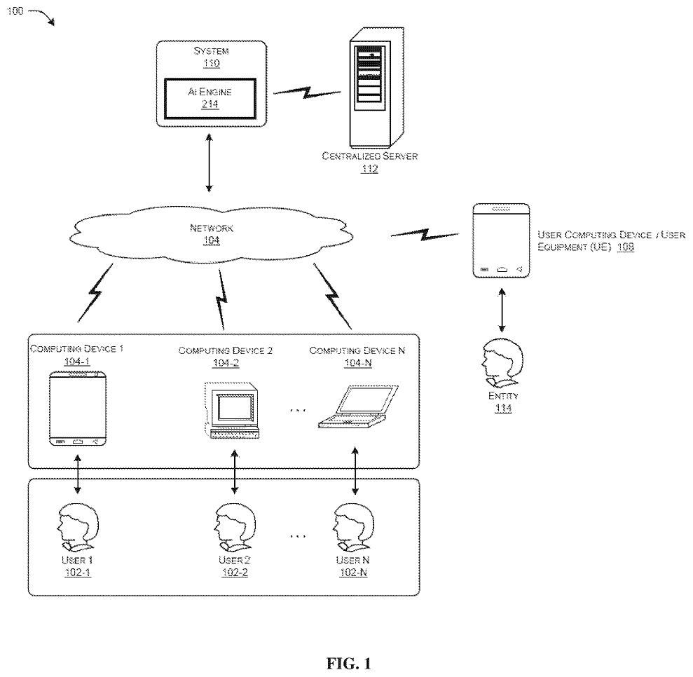

Referring to FIG. 1 that illustrates an exemplary network architecture (100) in which or with which system (110) of the present disclosure can be implemented, in accordance with an embodiment of the present disclosure. As illustrated, the exemplary architecture (100) includes a system (110) equipped with an artificial intelligence (AI) engine (214) for identification of a plurality of medical entities from a medical domain and rendering immediate online diagnostic solutions to a plurality of users (102-1, 102-2, . . . 102-n) (hereinafter interchangeably referred as user; and collectively referred to as users 102). Each user may be associated with at least one computing device (104-1, 104-2, . . . 104-n) (hereinafter interchangeably referred as a smart computing device or audio device; and collectively referred to as 104). The users (102) may interact with the system (110) by using their respective computing device (104), wherein the computing device (104) and the system (110) may communicate with each other over a network (106). The system (110) may be associated with a centralized server (112). Examples of the computing devices (104) can include, but are not limited to, a computing device (104) associated with healthcare and hospital-based assets, a smart phone, a portable computer, a personal digital assistant, a handheld phone and the like.

The system (110) may be further operatively coupled to a second computing device (108) (also referred to as the user computing device or user equipment (UE) hereinafter) associated with an entity (114). The entity (114) may include a company, a hospital, an organisation, a university, a lab facility, a business enterprise, or any other secured facility associated with health care research and related functionalities. In some implementations, the system (110) may also be associated with the UE (108). The UE (108) can include a handheld device, a smart phone, a laptop, a palm top and the like. Further, the system (110) may also be communicatively coupled to the one or more first computing devices (104) via a communication network (106).

Further, the network (106) can be a wireless network, a wired network, a cloud or a combination thereof that can be implemented as one of the different types of networks, such as Intranet, BLUETOOTH, MQTT Broker cloud, Local Area Network (LAN), Wide Area Network (WAN), Internet, and the like. Further, the network 106 can either be a dedicated network or a shared network. The shared network can represent an association of the different types of networks that can use variety of protocols, for example, Hypertext Transfer Protocol (HTTP), Transmission Control Protocol/Internet Protocol (TCP/IP), Wireless Application Protocol (WAP), and the like. In an exemplary embodiment, the network 104 can be an HC-05 Bluetooth module which is an easy to use Bluetooth SPP (Serial Port Protocol) module, designed for transparent wireless serial connection setup.

According to various embodiments of the present disclosure, the system 110 can provide for an Artificial Intelligence (AI) based automatic medical attribute detection, identification and input generation by using signal processing analytics. In an illustrative embodiment, the speech processing AI techniques can include, but not limited to, any or a combination of machine learning (referred to as ML hereinafter), deep learning (referred to as DL hereinafter) using concepts of neural network techniques.

In an aspect, the system (110) can receive an input corresponding to an input query pertaining to one or more symptoms associated with a disease. The system (110) may then extract a first set of attributes from the received input query, the first set of attributes pertaining to the one or more symptoms of the disease and further extract a second set of attributes from the received input query, the second set of attributes pertaining to demographic conditions and location. Based on the extracted first and second attributes, the Al engine (214) may map the one or more symptoms with a set of parameters. In an embodiment, the set of parameters may be determined by searching a medical knowledgebase for similarity of the set of parameters with the one or more symptoms. The medical knowledgebase may include static medical resources, medical databases, online medical resources, peer reviewed journals and the like for a target disease. The AI engine (214) may further be configured to diagnose the disease based on the mapped one or more symptoms with the set of parameters associated with the target disease.

In an exemplary embodiment, the system (110) may be configured to collect various points of data chosen by a plurality of medical experts to gather, refine and enrich the medical knowledgebase.

In an exemplary embodiment, the system (110) may be coupled to an interface unit configured to display a plurality of information associated with the diagnosed disease.

In an exemplary embodiment, the system (110) may perform a feedback analysis executed by the plurality of medical experts (interchangeably referred to as the medical experts). The medical expert may conduct a plurality of triages mimicking various clinical scenarios. Each triage assumption includes the target disease, age, gender, symptoms and context as may be applicable to that clinical scenario. In an exemplary embodiment, the one or more symptoms may include the initial symptoms (presenting complaints) and symptom questions put forth by the system (derived symptoms).

In an exemplary embodiment, after the plurality of triages are simulated by the medical experts, the journey is reviewed to find out the abnormalities or medically incorrect responses generated by the system. This exercise is iteratively performed to improve overall accuracy. These comprise the feedback signals from the triages.

In an exemplary embodiment, using the feedback signals, improvements and fixes may be found in the form of knowledge features. Ideally a feedback signal points to a certain feature of the knowledge graph which can be updated. In case the feature is not part of the knowledge graph already, the schema will be updated and reviewed to add the new feature in the knowledge graph. This exercise of knowledge feature review involves extensively the medical experts, knowledge engineering experts and the algorithm developers to analyze, review and approve the features before adding into the baseline medical knowledge by the medical experts.

In an exemplary embodiment, the system (110) may provide a knowledge graph modelling to represent the interconnections between primary entity nodes in the form of curated weighted edges, in order to improvise the differential diagnosis. The knowledge design allows for modelling dependencies between symptom attributes and symptom-attribute values further providing a possibility to ask conditional questions and the graph may include more than 20,000 “medically curated units of knowledge” with a high coverage with more than 1000 most common diseases and more than a thousand most common symptoms and their attributes. And a complex graph with more than a billion possible connections.

In an exemplary embodiment, the system (110) may allow for factoring in other dimensions of medical knowledge (contexts) whose dynamic nature works to augment the accuracy of diagnosis prediction. For example, the context may be looked at in at least two ways by the system: group specific and disease specific. The context may include but is not limited to demographic data, climate conditions, geolocation, lifestyle parameters, medical history, surgical history, drug history, laboratory and imaging tests, occupation, addiction(s) or habit(s) and the like. The entire corpus of medical knowledge is dynamically evolving and is capable of scaling exponentially. The knowledge graph described herein is coupled with appropriate visual representations of the encoded medical knowledge in a way that will be easily and accurately comprehensible to the patient. In addition to text representation, this also takes the form of:

Clickable humanoid representations. For example, abdominal pain is represented by interactive humanoid representation with the 9 segments of the abdomen, and the user may choose the part that correctly represents the part that she/he has the pain at. This representation allows for a more intuitive dialogue between the user and the system.
Color-coded scales, which make intuitive sense to the patient. For example, Intensity of pain is represented by a graded slider, on which the user can choose the placement that most faithfully replicates the intensity of pain.
Carousel of Images: In case of symptoms that have highly heterogencous presentations, a series of images is presented to the user, intended to cover a majority of the common presentations of that symptom.
Static images and GIFs (Graphics Interchange Format): These have been used to further enhance the possibilities in which symptoms can be asked to a user.

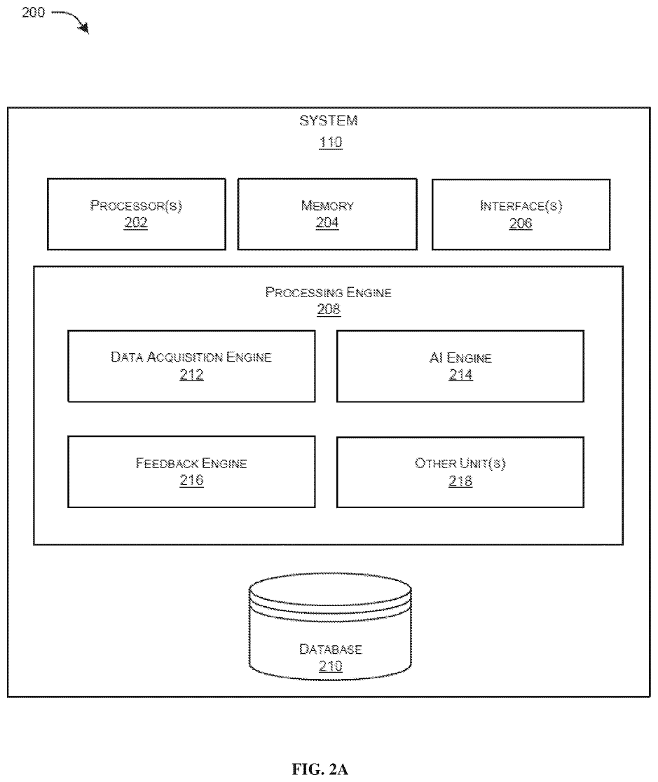

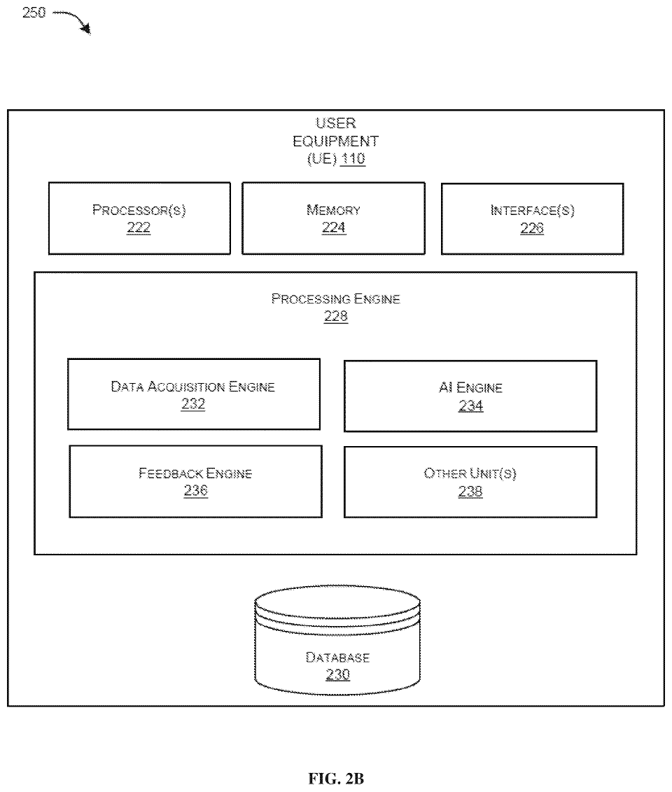

FIG. 2A illustrates an exemplary representation (200) of system (110), in accordance with an embodiment of the present disclosure.

In an aspect, the system (110) may comprise one or more processor(s) (202). The one or more processor(s) (202) may be implemented as one or more microprocessors, microcomputers, microcontrollers, digital signal processors, central processing units, logic circuitries, and/or any devices that process data based on operational instructions. Among other capabilities, the one or more processor(s) (202) may be configured to fetch and execute computer-readable instructions stored in a memory (204) of the system (110). The memory (204) may be configured to store one or more computer-readable instructions or routines in a non-transitory computer readable storage medium, which may be fetched and executed to create or share data packets over a network service. The memory (204) may comprise any non-transitory storage device including, for example, volatile memory such as RAM, or non-volatile memory such as EPROM, flash memory, and the like.

In an embodiment, the system (110) may include an interface(s) 206. The interface(s) 206 may comprise a variety of interfaces, for example, interfaces for data input and output devices, referred to as I/O devices, storage devices, and the like. The interface(s) 206 may facilitate communication of the system (102). The interface(s) 206 may also provide a communication pathway for one or more components of the centralized server (112). Examples of such components include, but are not limited to, processing engine(s) 208 and a database 210.

The processing engine(s) (208) may be implemented as a combination of hardware and programming (for example, programmable instructions) to implement one or more functionalities of the processing engine(s) (208). In examples described herein, such combinations of hardware and programming may be implemented in several different ways. For example, the programming for the processing engine(s) (208) may be processor executable instructions stored on a non-transitory machine-readable storage medium and the hardware for the processing engine(s) (208) may comprise a processing resource (for example, one or more processors), to execute such instructions. In the present examples, the machine-readable storage medium may store instructions that, when executed by the processing resource, implement the processing engine(s) (208). In such examples, the system (110) may comprise the machine-readable storage medium storing the instructions and the processing resource to execute the instructions, or the machine-readable storage medium may be separate but accessible to the system (110) and the processing resource. In other examples, the processing engine(s) (208) may be implemented by electronic circuitry.

The processing engine (208) may include one or more engines selected from any of a data acquisition (210), an artificial intelligence (AI) engine (214), a feedback analysis engine (216) and other engines (218).

In an exemplary embodiment, the data acquisition engine (210) can receive an input corresponding to an input query pertaining to one or more symptoms associated with a disease. The AI engine (214) may then extract a first set of attributes from the received input query, the first set of attributes pertaining to the one or more symptoms of the disease and further extract a second set of attributes from the received input query, the second set of attributes pertaining to demographic conditions and location. Based on the extracted first and second attributes, the AI engine (214) may map the one or more symptoms with a set of parameters. In an embodiment, the set of parameters may be determined by searching a medical knowledgebase for similarity of the set of parameters with the one or more symptoms. The medical knowledgebase may include static medical resources, medical databases, online medical resources, peer reviewed journals and the like for a target disease. The Al engine (214) may further be configured to diagnose the disease based on the mapped one or more symptoms with the set of parameters associated with the target disease.

In an exemplary embodiment, the feedback analysis engine (216) may perform a feedback analysis executed by the plurality of medical experts (interchangeably referred to as the medical experts). The medical expert may conduct a plurality of triages mimicking various clinical scenarios. Each triage assumption includes the target disease, age, gender, symptoms and context as may be applicable to that clinical scenario. In an exemplary embodiment, the one or more symptoms may include the initial symptoms (presenting complaints) and symptom questions put forth by the system (derived symptoms).

In an exemplary embodiment, after the plurality of triages are simulated by the medical experts, the journey is reviewed to find out the abnormalities or medically incorrect responses generated by the system. This exercise is iteratively performed to improve overall accuracy. These comprise the feedback signals from the triages.

In an exemplary embodiment, feedback analysis engine (216) using the feedback signals, may determine improvements and fixes in the form of knowledge features. Ideally a feedback signal points to a certain feature of the knowledge graph which can be updated. In case the feature is not part of the knowledge graph already, the schema will be updated and reviewed to add the new feature in the knowledge graph. This exercise of knowledge feature review involves extensively the medical experts, knowledge engineering experts and the algorithm developers to analyze, review and approve the features before adding into the baseline medical knowledge by the medical experts.

FIG. 2B illustrates an exemplary representation (250) of the user equipment (UE) (108), in accordance with an embodiment of the present disclosure. In an aspect, the UE (108) may comprise a processor (222). The more processor (222) may be implemented as one or more microprocessors, microcomputers, microcontrollers, digital signal processors, central processing units, logic circuitries, and/or any devices that process data based on operational instructions. Among other capabilities, the processor(s) (222) may be configured to fetch and execute computer-readable instructions stored in a memory (224) of the UE (108). The memory (224) may be configured to store one or more computer-readable instructions or routines in a non-transitory computer readable storage medium, which may be fetched and executed to create or share data packets over a network service. The memory (224) may comprise any non-transitory storage device including, for example, volatile memory such as RAM, or non-volatile memory such as EPROM, flash memory, and the like.

In an embodiment, the UE (108) may include an interface(s) 206. The interface(s) 206 may comprise a variety of interfaces, for example, interfaces for data input and output devices, referred to as I/O devices, storage devices, and the like. The interface(s) 206 may facilitate communication of the UE (108). Examples of such components include, but are not limited to, processing engine(s) 228 and a database (230).

The processing engine(s) (228) may be implemented as a combination of hardware and programming (for example, programmable instructions) to implement one or more functionalities of the processing engine(s) (228). In examples described herein, such combinations of hardware and programming may be implemented in several different ways. For example, the programming for the processing engine(s) (228) may be processor executable instructions stored on a non-transitory machine-readable storage medium and the hardware for the processing engine(s) (228) may comprise a processing resource (for example, one or more processors), to execute such instructions. In the present examples, the machine-readable storage medium may store instructions that, when executed by the processing resource, implement the processing engine(s) (228). In such examples, the UE (108) may comprise the machine-readable storage medium storing the instructions and the processing resource to execute the instructions, or the machine-readable storage medium may be separate but accessible to the UE (108) and the processing resource. In other examples, the processing engine(s) (228) may be implemented by electronic circuitry.

The processing engine (228) may include one or more engines selected from any of a data acquisition (230), an artificial intelligence (AI) engine (234), a feedback analysis engine (236) and other engines (238).

In an exemplary embodiment, the data acquisition engine (230) can receive an input corresponding to an input query pertaining to one or more symptoms associated with a disease. The AI engine (234) may then extract a first set of attributes from the received input query, the first set of attributes pertaining to the one or more symptoms of the disease and further extract a second set of attributes from the received input query, the second set of attributes pertaining to demographic conditions and location. Based on the extracted first and second attributes, the Al engine (234) may map the one or more symptoms with a set of parameters. In an embodiment, the set of parameters may be determined by searching a medical knowledgebase for similarity of the set of parameters with the one or more symptoms. The medical knowledgebase may include static medical resources, medical databases, online medical resources, peer reviewed journals and the like for a target disease. The AI engine (234) may further be configured to diagnose the disease based on the mapped one or more symptoms with the set of parameters associated with the target disease.

In an exemplary embodiment, the feedback analysis engine (236) may perform a feedback analysis executed by the plurality of medical experts (interchangeably referred to as the medical experts). The medical expert may conduct a plurality of triages mimicking various clinical scenarios. Each triage assumption includes the target disease, age, gender, symptoms and context as may be applicable to that clinical scenario. In an exemplary embodiment, the one or more symptoms may include the initial symptoms (presenting complaints) and symptom questions put forth by the system (derived symptoms).

In an exemplary embodiment, after the plurality of triages are simulated by the medical experts, the journey is reviewed to find out the abnormalities or medically incorrect responses generated by the system. This exercise is iteratively performed to improve overall accuracy. These comprise the feedback signals from the triages.

In an exemplary embodiment, feedback analysis engine (236) using the feedback signals, may determine improvements and fixes in the form of knowledge features. Ideally a feedback signal points to a certain feature of the knowledge graph which can be updated. In case the feature is not part of the knowledge graph already, the schema will be updated and reviewed to add the new feature in the knowledge graph. This exercise of knowledge feature review involves extensively the medical experts, knowledge engineering experts and the algorithm developers to analyze, review and approve the features before adding into the baseline medical knowledge by the medical experts.

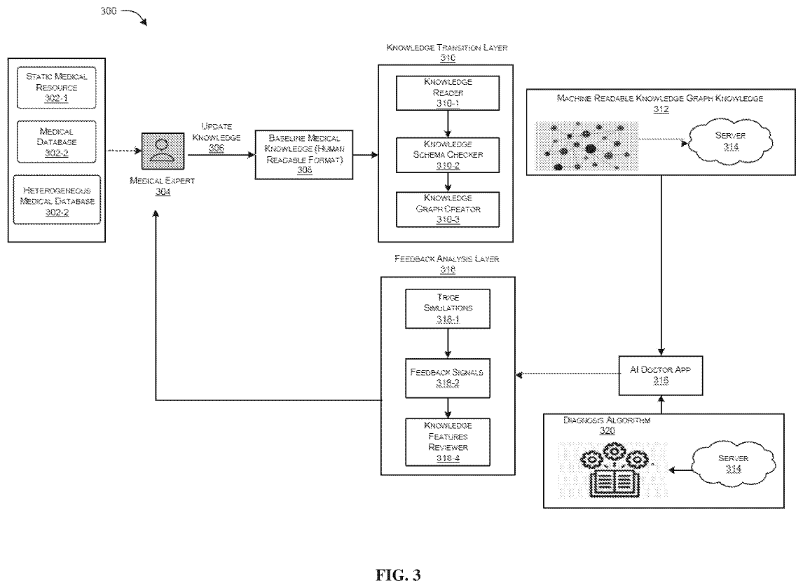

FIG. 3 illustrates an exemplary block diagram (300) representation of the proposes system in accordance with an embodiment of the present disclosure. As illustrated, in an aspect, the system may include static medical resources (302-1), medical databases (302-2), a heterogenous medical database (302-3) including but not limited to accepted online medical resources, peer reviewed journals and the like are the various points of data collection chosen by the medical experts to gather, refine and enrich the medical knowledge.

In an exemplary embodiment, the system (110) may include a baseline medical knowledge (308) is the human readable format stored in the Microsoft excel tables which is the first storage point used by the medical experts. The excel has a defined schema and the data is entered as per the schema which can be updated each time the knowledge features are reviewed to improve the medical knowledge representation.

In an exemplary embodiment, the system (110) may include knowledge transition layer (310) that may include knowledge reader (310-1), a Knowledge schema checker/validator (310-2), a Knowledge graph creator (310-3). This is the first module of the knowledge transition layer. The knowledge reader (310-1) takes the excel tables as the input and reads all the tabs, rows and columns within the tables and converts it into the data frames which are easy to query and compute. The Knowledge schema checker/validator (310-2) is the second module of the knowledge transition layer. The knowledge schema checker parses all the tabs, rows and columns in the data frames that are read by the knowledge reader to verify the format and schema of the data entered by the medical experts. E.g.: The age format should be an integer in the excel tables. If the age format is stored in string, then the knowledge schema checker will not let the knowledge transition layer proceed to further execution steps. The Knowledge graph creator (310-3) is the third and final module in the knowledge transition layer. Once the data stored by the medical experts is read, converted, parsed and validated by the first two modules of the knowledge transition layer, the knowledge graph creator interconnects various rows, columns and tabs as per the pre-defined process and generates the medical knowledge graph.

The system may further include an AI-Doctor (316) application that may be started by the medical experts which uses the medical knowledge graph created by the knowledge transition layer and the required diagnosis algorithms from the server.

The system may include a Feedback analysis layer (318) that may include triage simulations (318-1), Feedback signals (318-2), Knowledge features reviewer (318-3). The triage simulations (318-2) which is the first step executed by the medical experts in the feedback analysis layer. The medical expert conducts multiple triages mimicking various clinical scenarios. Each triage assumption includes the target disease, age, gender, symptoms and context as may be applicable to that clinical scenario. Symptoms include the initial symptoms (presenting complaints) and symptom questions put forth by the system (follow up symptoms). The Feedback signals (318-2) is the second step of the feedback analysis layer.

After the triages are simulated by the medical experts, the journey is reviewed to find out the abnormalities or medically incorrect responses generated by the system. This exercise is iteratively performed to improve overall accuracy. These comprise the feedback signals from the triages. The Knowledge features reviewer (318-3) is the third and final step of the feedback analysis layer. Using the feedback signals in the previous step, the improvements and fixes are found in the form of knowledge features. Ideally a feedback signal points to a certain feature of the knowledge graph which can be updated. In case the feature is not part of the knowledge graph already, the schema will be updated and reviewed to add the new feature in the knowledge graph. This exercise of knowledge feature review involves extensively the medical experts, knowledge engineering experts and the algorithm developers to analyze, review and approve the features before adding into the baseline medical knowledge by the medical experts.

In an exemplary embodiment, Feedback signals include:
Target disease(s)∉top k predicted disease(s)
confidence_score (target disease) is not correct
Δ (score (target disease), score (differential diagnosis)) is incorrect
Incorrect diseases in top ranks
Incorrect order of differential diagnosis
Diseases with incorrect body system
Diseases with incorrect criticality
Incorrect symptoms asked
Incorrect symptom attributes asked
Less questions asked
More questions asked

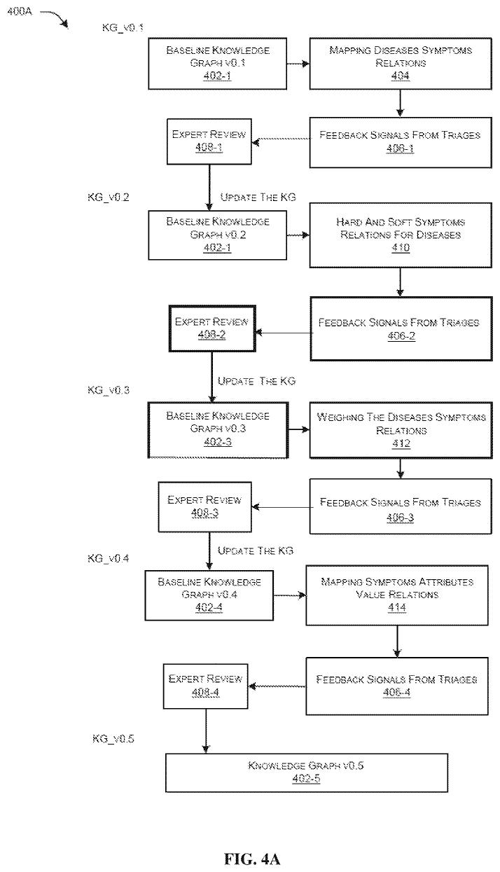

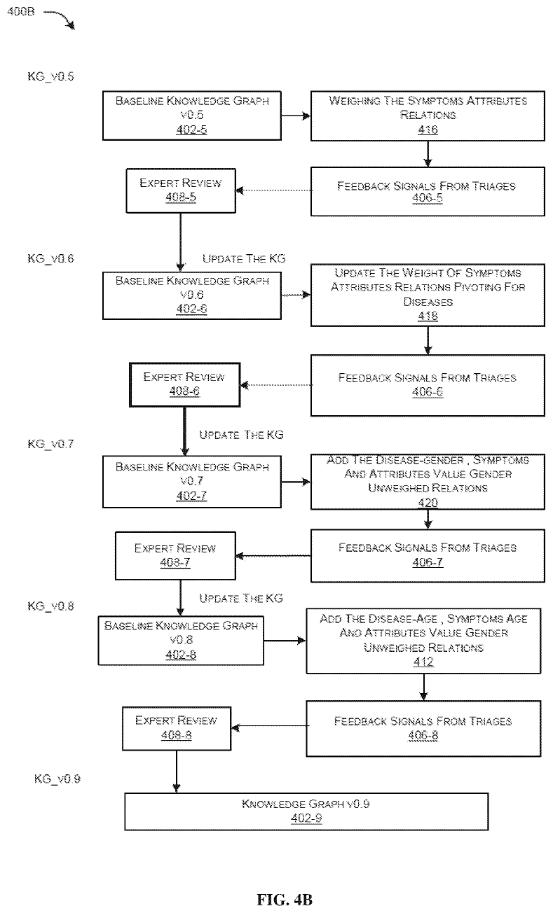

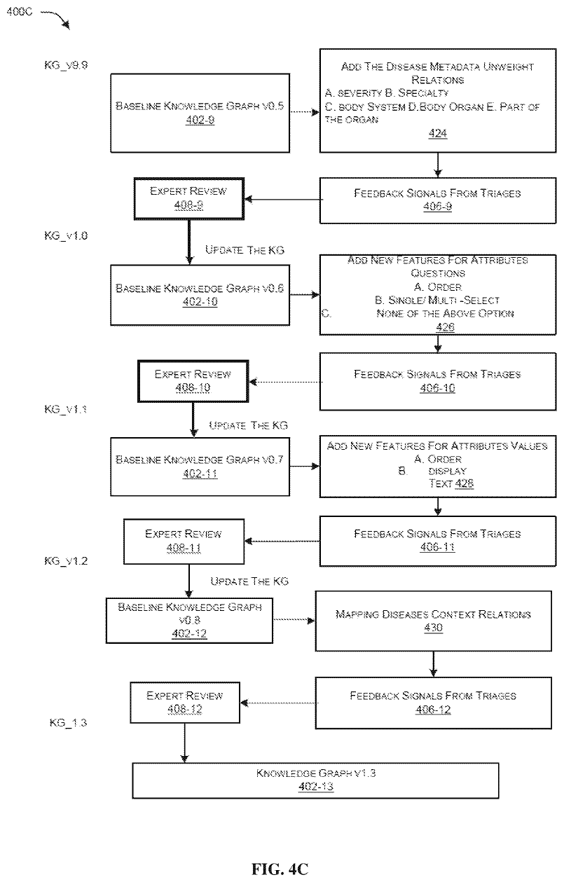

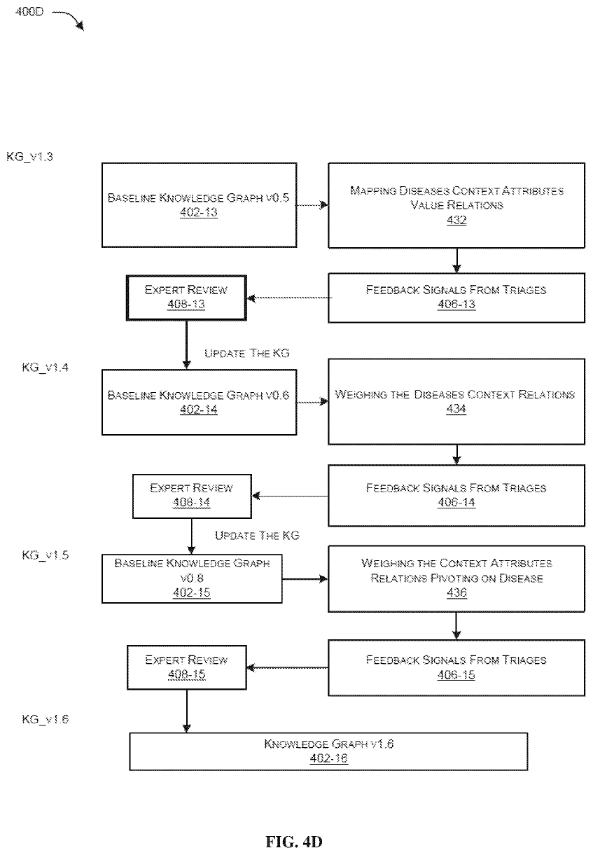

FIGS. 4A-4D illustrates exemplary flow diagram representation (400) of the proposed method, in accordance with an embodiment of the present disclosure. As illustrated in FIGS. 4A-4D, the knowledge graph may be created with a base knowledge and extended to new features in a phase-wise manner which are shown in different versions. Each phase or version follows at least three steps to complete one iteration that may include the steps of
Adding new feature in the baseline knowledge graph (402)
Running triage simulations to find the feedback signals (406)
Reviewing the feedback signals to update the knowledge graph to next version (408)

However, this process focuses more on the features added in each phase rather than how the features are identified.

In an exemplary embodiment, a knowledge graph 0.1 (402-1) may include disease-Symptom relation (404) where each disease node is mapped to one or more symptom nodes in the knowledge graph. The relation between each disease and symptom is an unweighted edge which means all the symptoms are equally important for a disease.

In an exemplary embodiment, a knowledge graph 0.2 (402-2) may provide attributes on hard or soft symptoms of disease (410) where each disease and symptom relation are bucketed into two types as hard or soft based on the symptom nature (importance or frequency or rarity). There could be single or multiple hard or soft symptoms associated with a disease and in case of multiple symptoms, all the symptoms are equally important in terms of hardness or softness.

In an exemplary embodiment, a knowledge graph 0.3 (402-3) may provide attributes on weighing the disease-symptom relation (412) that may include giving a disease-symptom relation on a quantitative weight or bucket based on the importance, hardness and softness of the symptom. The bucket can ranges from, for example, 1 to 4 where the hard symptoms fall in buckets 1 or 2 and soft symptoms fall in buckets 3 or 4. The symptoms are divided into 4 different buckets based on the below criteria:
I. Bucket 1: Classical primary symptom
II. Bucket 2: Classical salient symptom
III. Bucket 3: Supportive symptom
IV. Bucket 4: Uncommon symptom

In an exemplary embodiment, a knowledge graph 0.3 (402-4) may provide Symptom-Attribute-Value relations (414) where the symptoms are not the tail-end nodes for a disease. To understand more details of a symptom, its attributes are added and for each attribute its possible values are added. All the attributes are treated equally for a symptom, hence the symptom-attribute relation is unweighted. All the values are treated equally for an attribute, hence the attribute-value relation is unweighted.

In an exemplary embodiment, a knowledge graph 0.5 (402-5) may provide attributes on weighing the symptom-attribute relations (416) such as the symptoms have quantitative relation with a disease in the form of bucket, the attribute also has a quantitative relation with its symptom. The attributes are divided into one of the 4 buckets based on its importance for a symptom but independent of disease. Each bucket can have single or multiple attributes.

In an exemplary embodiment, a knowledge graph 0.6 (402-6) may provide attributes on weighing the symptom-attribute pivoting on disease (418). The attribute bucket is updated by its importance with a symptom as well as disease. The role of attribute can change both with disease and without disease which is a unique and strong value add for quick diagnosis.

In an exemplary embodiment, a knowledge graph 0.7 (402-7) may provide Gender relation (420) attributes. For example, the diseases, symptoms and attribute values can be dependent on the gender of the patient as a hard filter in most of the medical cases and help narrow down the possible diagnosis. The appropriate gender (Male/Female/Both) is mapped with an unweighted relation for all the diseases, symptoms and attribute values. For example, “Uterine Fibroid” is possible in female patients only.

In an exemplary embodiment, a knowledge graph 0.8 (402-8) may provide age range relation attributes (422). For example, the diseases, symptoms and attribute values can be dependent on the age range of the patient as a hard or soft filter in most of the medical cases and help narrow down the possible diagnosis. The appropriate age range is mapped with an unweighted relation for all the diseases, symptoms and attribute values. The minimum and maximum ages are defined separately in the table. E.g.; “Benign prostate hypertrophy” is unlikely in younger patients.

In an exemplary embodiment, a knowledge graph 0.9 (402-9) may provide attributes on disease-metadata relations (424). For example, the disease can be diagnosed by the symptoms but its metadata can help in quick and accurate diagnosis. The metadata added are disease severity, specialty, body system, body organ and part of organ. All metadata has an unweighted and qualitative relation with its disease. Body system, body organ and part of organ help in better diagnosis. Severity and specialty help in post-diagnosis guidance to the patient which is crucial.

In an exemplary embodiment, a knowledge graph 1.0 (402-10) may provide attributes on enriching the attributes (426). For example, the attributes have values which can be single or multiselect and are pre-defined. Despite some attributes having exhaustive values, the user might not find the value most applicable to them, hence “none of the above” has been added as a default value. Attributes have also been given an order. This is done to ensure good dialogue.

In an exemplary embodiment, a knowledge graph 1.1 (402-11) may provide attributes on enriching the attribute values (428). For example, attributes have also been given an order. This is done to ensure good dialogue. E.g.: Fever “range” looks acceptable when “Mild”, “Moderate”, “High” & “Hyperpyrexia” is the sequence. The display text of each value is converted from medical terminology to non-medical terms which helps the user understand it better. E.g.: Fever duration: “Less than 2 days” is more understandable than “Acute”.

In an exemplary embodiment, a knowledge graph 1.2 (402-12) may provide attributes on disease-context relation (430). For example, each disease node is mapped to one or more context nodes in the knowledge graph. The relation between each disease and context is an unweighted edge which means all the contexts are equally important for a disease. For example, a Disease context relation is mapped to gender: Male/Female/Both.

In an exemplary embodiment, a knowledge graph 1.3 (402-13) may provide attributes on context-attribute-value relations (432). For example, the contexts are not the tail-end nodes for a disease. To understand more details of a context, its attributes are added and for each attribute its values are added. All the attributes are treated equally for a context; hence the context-attribute relation is unweighted. All the values are treated equally for an attribute; hence the attribute-value relation is unweighted. Context-attributes values are also mapped to gender: Male/Female/Both. Context-attribute values have also been given an order for good dialogue flow.

In an exemplary embodiment, a knowledge graph 1.4 (402-14) may provide attributes on disease-context weights (434). For example, the Disease-Context relation is given a weight, which best reflects the contribution of that context for that particular disease. For example, alcohol context is weighted as bucket 1 for Alcoholic Liver Disease, reflecting its importance as utmost in the diagnosis of this disease. The bucket ranges from 1 to 4 where bucket 1 is for most important or very common contexts and bucket 4 is for least important or rare contexts.

In an exemplary embodiment, a knowledge graph 1.5 may provide attributes on Disease-Context-Attribute Weights (436). For example, the Context-Attribute relation is also weighted in a way which reflects the contribution of that attribute in the backdrop of that particular context and disease combination. Hence, the attributes have quantitative relation with its context and disease combination in the form of bucket similar to how the contexts have quantitative relation with diseases. Each attribute falls into one of the 4 buckets based on its importance for a context and disease combination. Each bucket can have single or multiple attributes.

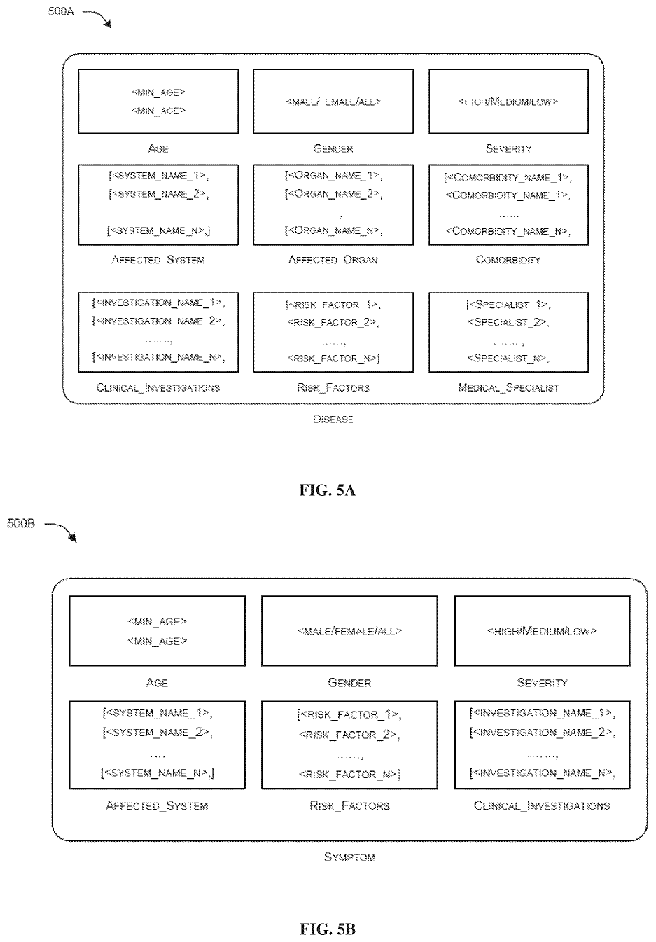

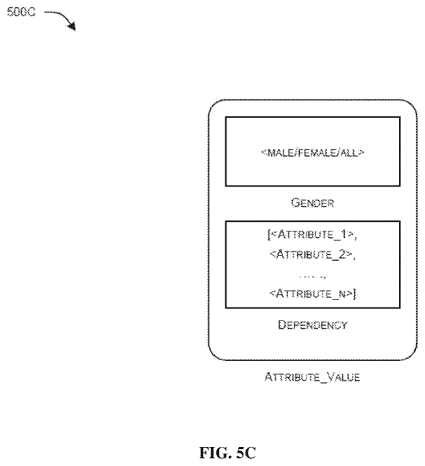

FIGS. 5A-5C illustrate exemplary representations of flow diagrams associated with a medical knowledgebase, in accordance with an embodiment of the present disclosure. FIG. 5A illustrates an exemplary representation of a knowledge base that has categories to store and sort data according to age, gender, severity of disease, affected system, affected organ, comorbidity, clinical investigations, risk factors, medical expert associated with a certain disease while FIG. 5B illustrates an exemplary representation of a knowledge base that has categories to store and sort data according to age, gender, severity of disease, affected system, affected organ, comorbidity, clinical investigations, risk factors, medical expert associated with one or more symptoms. FIG. 5C illustrates an exemplary representation of a knowledge base that has categories to store and sort data according to gender and dependency associated with an attribute value.

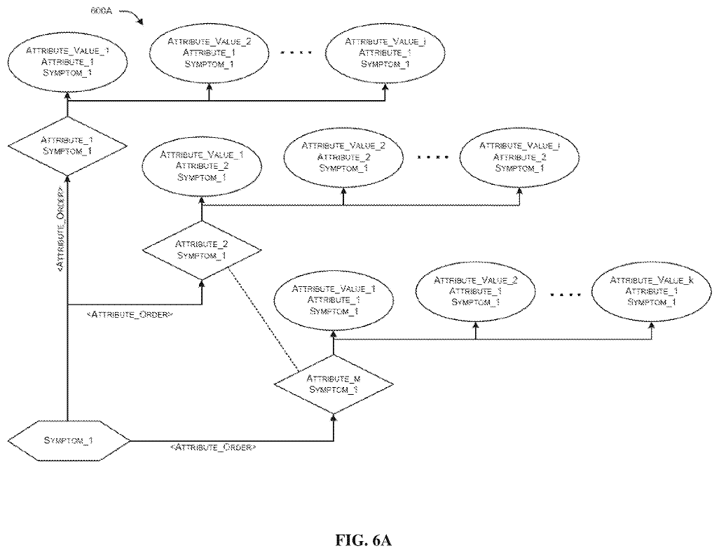

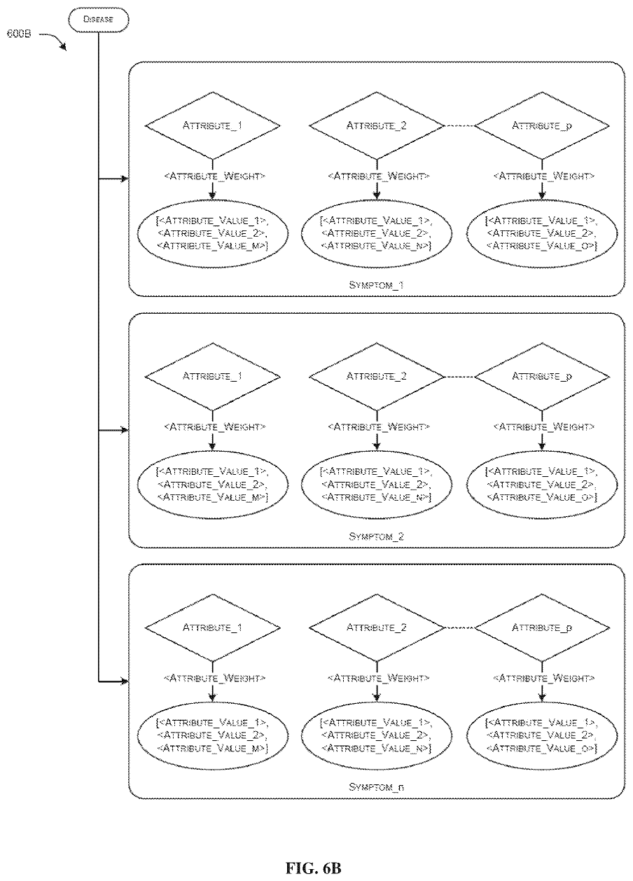

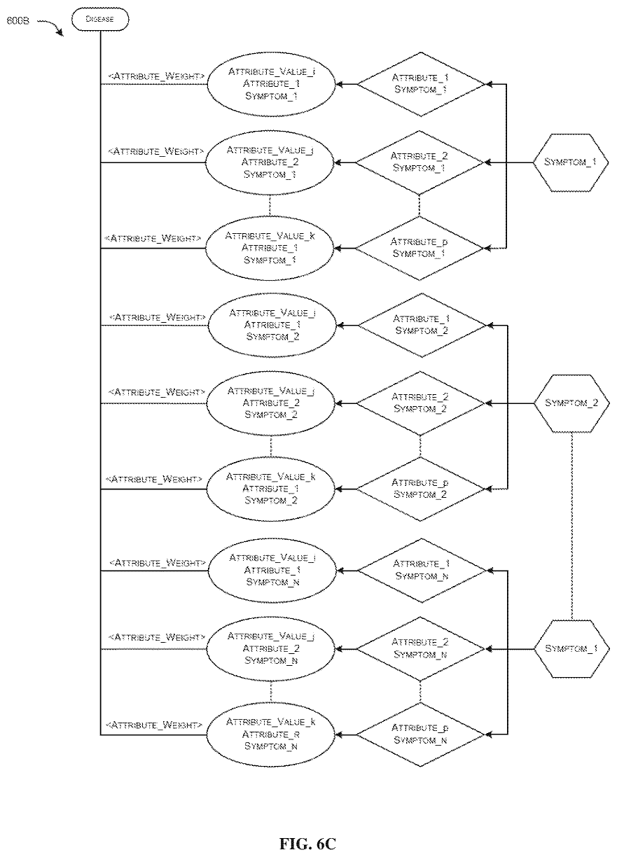

While FIGS. 6A-6C illustrate exemplary representations of flow diagrams associated with disease-Symptom view, in accordance with an embodiment of the present disclosure. FIG. 6A shows how from symptom 1, various attributes are obtained and mapped into the respective attribute values. As shown in FIG. 6B, a first attribute is associated with a first weigh to obtain attribute values for a first symptom. This process is done for pth number of attributes for the first symptom. Similarly, the process is done for an nth symptom. FIG. 6C shows how from symptom 1, symptom 2 and symptom 3 are mapped with the respective attributes and their attribute values.

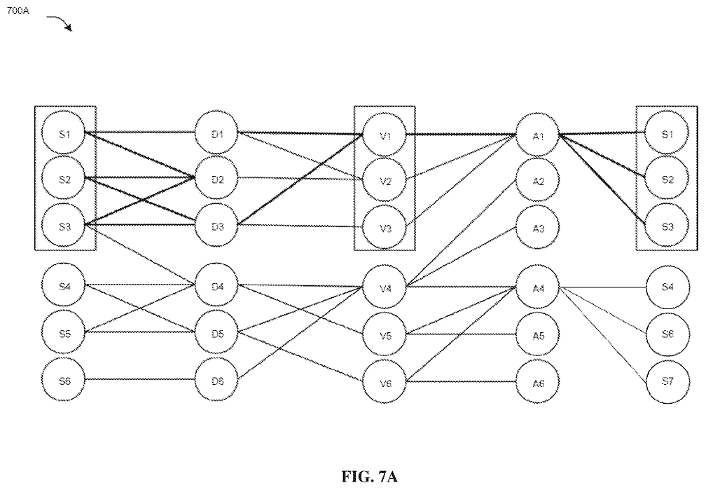

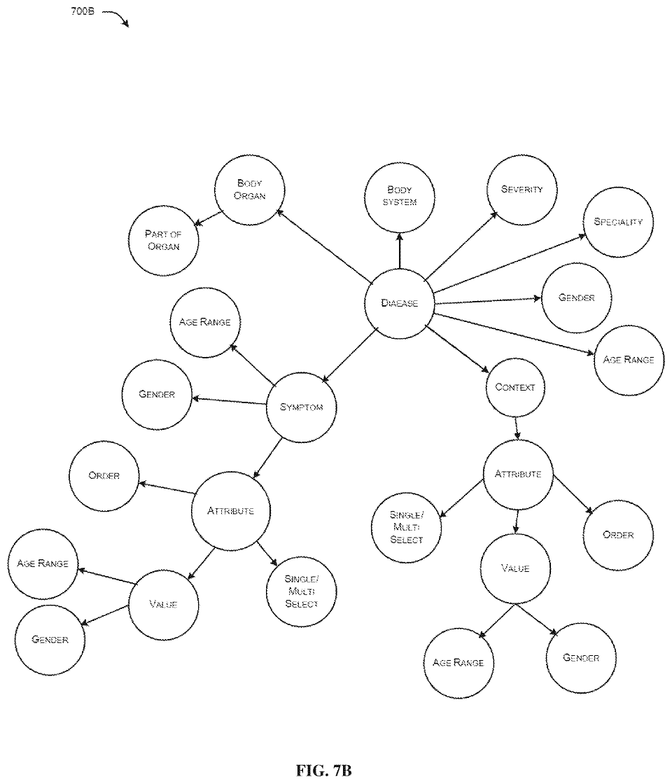

FIGS. 7A-7B illustrate exemplary tree diagram representation of diagnostics as service. As illustrated, Knowledge design allows for modelling dependencies between symptom attributes and symptom-attribute values further providing a possibility to ask conditional questions. Schema design allows for factoring in other contexts such as disease priors, risk factors, lab-tests, body organs, body systems etc. Inputs for the invention can also be taken from medical and health devices, including but not limited to Wearable IoT based devices like Health bands, Continuous Glucose Monitoring (CGM) devices, Drug Pumps (e.g. Insulin Pumps), Thermometer, Weighing Scale, Digital Stethoscope, Digital Sphygmomanometer (BP Apparatus), ECG Machine, Echo Machine, Spirometer, Pulse oximeter, Automated analyser, Glucometer, Blood-Gas Analyser, Ophthalmoscope, Otoscope, Imaging Modalities like, (but not limited to) X-Rays, Ultrasound, MRI, CT Scan, Doppler, PET Scan.

The overall knowledge representation or schema comprising of the following important entities
Disease
Symptom
Attribute
Value
Disease-Symptom weight
Symptom-Attribute weight
Symptom-Attribute Value weight
Disease-Context weight
Context-Attribute weight
Context-Attribute Value weight
Symptom-Attribute order (helps in dependent attributes)
Attribute-Value order
Attribute-single/multiselect
Attribute-Dependent attribute
Body System
Organ
Part of organ
Disease severity
Specialty
Disease-AgeRange relation
Symptom-AgeRange relation
Value-AgeRange relation
Disease-Gender relation
Symptom-Gender relation
Value-Gender relation
Context-Gender relation
Context-Attribute gender relation

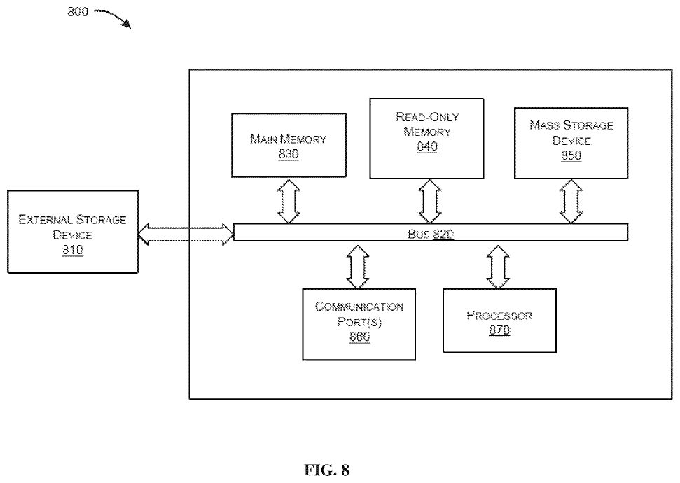

FIG. 8 illustrates an exemplary computer system in which or with which embodiments of the present invention can be utilized in accordance with embodiments of the present disclosure. As shown in FIG. 8, computer system 800 can include an external storage device 810, a bus 820, a main memory 830, a read only memory 840, a mass storage device 850, communication port 860, and a processor 870. A person skilled in the art will appreciate that the computer system may include more than one processor and communication ports. Processor 870 may include various modules associated with embodiments of the present invention. Communication port 860 may be chosen depending on a network to which computer system connects. Memory 830 can be Random Access Memory (RAM), or any other dynamic storage device commonly known in the art. Read-only memory 840 can be any static storage device(s). Mass storage 870 may be any current or future mass storage solution, which can be used to store information and/or instructions.

Bus 820 communicatively couples processor(s) 870 with the other memory, storage and communication blocks. Optionally, operator and administrative interfaces, e.g. a display, keyboard, joystick and a cursor control device, may also be coupled to bus 820 to support direct operator interaction with a computer system. Other operator and administrative interfaces can be provided through network connections connected through communication port 860. Components described above are meant only to exemplify various possibilities. In no way should the aforementioned exemplary computer system limit the scope of the present disclosure.

Thus, the present disclosure provides a unique and inventive solution for a schema design comprising of primary entities in the form of diseases and symptoms. Further, the system provides a knowledge graph modelling to represent the interconnections between the primary entity nodes in the form of curated weighted edges, design of various types of weights in order to improvise the differential diagnosis. The knowledge design allows for modelling dependencies between symptom attributes and symptom-attribute values further providing a possibility to ask conditional questions and the graph may include but not limited to 13000 highly curated “units of knowledge” with a high coverage with about 450 diseases and 800 symptoms and their attributes. And a complex graph with 1.63 billion maximum possible connections. The Schema design allows for factoring in other dimensions of medical knowledge (contexts) whose dynamic nature works to augment the accuracy of diagnosis prediction. Context is looked at at least two ways by the system: group specific and disease specific. The context may include but is not limited to demographic data, climate conditions, geolocation, lifestyle parameters, medical history, surgical history, drug history, laboratory and imaging tests, occupation, addiction(s) or habit(s) and the like. The entire corpus of medical knowledge is a dynamically evolving entity and is capable of being scaled exponentially. The knowledge graph described herein is coupled to appropriate visual representations of the encoded medical knowledge in a way that will be easily and accurately comprehensible to the patient. In addition to text representation, this also takes the form of: clickable humanoid representations, Color-coded scales, Carousel of Images, Static images and GIFs (Graphics Interchange Format) and the like.

While considerable emphasis has been placed herein on the preferred embodiments, it will be appreciated that many embodiments can be made and that many changes can be made in the preferred embodiments without departing from the principles of the invention. These and other changes in the preferred embodiments of the invention will be apparent to those skilled in the art from the disclosure herein, whereby it is to be distinctly understood that the foregoing descriptive matter to be implemented merely as illustrative of the invention and not as limitation.

A portion of the disclosure of this patent document contains material which is subject to intellectual property rights such as, but are not limited to, copyright, design, trademark, IC layout design, and/or trade dress protection, belonging to Jio Platforms Limited (JPL) or its affiliates (herein after referred as owner). The owner has no objection to the facsimile reproduction by anyone of the patent document or the patent disclosure, as it appears in the Patent and Trademark Office patent files or records, but otherwise reserves all rights whatsoever. All rights to such intellectual property are fully reserved by the owner.

### Advantages of the Present Disclosure

The present disclosure provides for a system and a method that includes more than 20,000 highly curated units of knowledge associated with diseases and their symptoms.

The present disclosure provides for a system and a method that facilitates coverage of more than a 1000 most common diseases and more than a 1000 most common symptoms and their attributes.

The present disclosure provides for a system and a method that facilitates complex graph with more than a billion possible connections.

The present disclosure provides for a schema design that allows for factoring in other dimensions of medical knowledge, called contexts, beyond diseases, symptoms, and symptom attributes whose combination with these primary entities fine-tunes and adapts the diagnosis by such parameter and further increases the accuracy of diagnosis prediction.

The present disclosure provides for a system and a method that is not limited to demographic data, climate conditions, geolocation, lifestyle parameters, medical history, surgical history, drug history, laboratory and imaging tests, occupation, addiction(s) or habit(s) etc.

The present disclosure provides for system and a method that is a dynamically evolving entity and is capable of being scaled exponentially.

The present disclosure provides for a system and a method that enables visual representations of the encoded medical knowledge in a way that will be easily and accurately comprehensible to the patient is a plurality of representations such as textual, clickable humanoid representations, Color-coded scales, Carousel of Images, Static images and GIFs (Graphics Interchange Format) and the like.

We claim:

1. A system (110) for providing enhanced medical symptom checker, said system (110) comprising;
one or more processors (202) operatively coupled to a plurality of first computing devices (104), the one or more processors (202) coupled with a memory (204), wherein said memory (204) stores instructions which when executed by the one or more processors (202) causes said system (110) to:
receive an input query from one or more users associated with the plurality of first computing devices, said input query pertaining to one or more symptoms associated with a disease;
extract a first set of attributes from the received input query, wherein the first set of attributes pertain to the one or more symptoms of the disease;
extract a second set of attributes from the received input query, the second set of attributes pertain to demographic conditions and location;
based on the extracted first and second attributes, map the one or more symptoms with a set of parameters, wherein the set of parameters are determined by searching a medical knowledge-base for similarity of the set of parameters in the one or more symptoms, wherein the medical knowledge-base comprises static medical resources, medical databases, online medical resources, and peer reviewed journals for a target disease; and,
diagnose the disease based on the mapped one or more symptoms with the set of parameters associated with the target disease.

2. The system as claimed in claim 1, wherein the system is further configured to collect a plurality of points of data chosen by a plurality of medical experts to gather, refine and enrich the medical knowledgebase.

3. The system as claimed in claim 1, wherein the system (110) is further coupled to an interface unit configured to display a plurality of information associated with the diagnosed disease.

4. The system as claimed in claim 1, wherein the system (110) is further configured to:
perform a feedback analysis executed by the plurality of medical experts, wherein the feedback analysis is based on a plurality of triages performed by the plurality of medical experts mimicking a plurality of clinical scenarios, wherein each said triage includes the target disease, age, gender, one or more symptoms and context applicable to the plurality of clinical scenarios.

5. The system as claimed in claim 1, wherein the system is configured to generate one or more derived symptoms based on one or more symptom questions and an initial set of complaints provided by a user.

6. The system as claimed in claim 1, wherein the one or more symptoms includes the set of initial symptoms and the one or more derived symptoms.

7. The system as claimed in claim 1, wherein the system is further configured to find out a plurality of abnormalities or medically incorrect responses generated by the system.

8. The system as claimed in claim 1, wherein the system is further configured to
improve and fix a knowledge graph comprising one or more knowledge features associated with the medical knowledgebase;
update the one or more knowledge features in the knowledge graph;
add a new feature to the knowledge graph.

9. The system as claimed in claim 8, wherein the knowledge graph is coupled with appropriate visual representations of a medical knowledge that is easily and accurately comprehensible to the user.

10. The system as claimed in claim 1, wherein the system is further configured to allow for factoring in a plurality of dimensions of medical knowledge whose dynamic nature works to augment the accuracy of diagnosis prediction.

11. The system as claimed in claim 1, wherein the system is further coupled to a user interface configured with an intuitive dialogue between the user and the system.

12. The system as claimed in claim 11, wherein the user interface is configured with one or more clickable interactive humanoid representations, wherein the user chooses one or more clickable interactive humanoid representations that represent the part that the user has the pain at.

13. A user equipment (UE) for providing enhanced medical symptom checker, said UE comprising;
a processor (222) and a receiver operatively coupled to a plurality of first computing devices (104), the processor (222) coupled with a memory (224), wherein said memory (224) stores instructions which when executed by the processor (222) causes said UE (108) to:
receive, by the receiver, an input query from one or more users associated with the plurality of first computing devices, said input query pertaining to one or more symptoms associated with a disease;
extract, by the processor (222), a first set of attributes from the received input query, wherein the first set of attributes pertain to the one or more symptoms of the disease;
extract, by the processor (222), a second set of attributes from the received input query, the second set of attributes pertain to demographic conditions and location;
based on the extracted first and second attributes, map, by the processor (222), the one or more symptoms with a set of parameters, wherein the set of parameters are determined by searching a medical knowledgebase for similarity of the set of parameters in the one or more symptoms, wherein the medical knowledgebase comprises static medical resources, medical databases, online medical resources, and peer reviewed journals for a target disease; and,
diagnose, by the processor (222), the disease based on the mapped one or more symptoms with the set of parameters associated with the target disease.

14. The UE as claimed in claim 13, wherein the UE is equipped with a user interface configured with an intuitive dialogue between the user and the UE.

15. The UE as claimed in claim 13, wherein the user interface is configured with one or more clickable interactive humanoid representations, wherein the user chooses one or more clickable interactive humanoid representations that represent the part that the user has the pain at.

16. A method for providing enhanced medical symptom checker, said method comprising the steps of:
receiving, by one or more processors (202), an input query from one or more users associated with the plurality of first computing devices, said input query pertaining to one or more symptoms associated with a disease;
extracting, by the one or more processors (202), a first set of attributes from the received input query, wherein the first set of attributes pertain to the one or more symptoms of the disease;
extracting, by the one or more processors (202), a second set of attributes from the received input query, the second set of attributes pertain to demographic conditions and location;
based on the extracted first and second attributes, mapping, by the one or more processors (202), the one or more symptoms with a set of parameters, wherein the set of parameters are determined by searching a medical knowledgebase for similarity of the set of parameters in the one or more symptoms, wherein the medical knowledgebase comprises static medical resources, medical databases, online medical resources, and peer reviewed journals for a target disease; and,
diagnosing, by the one or more processors (202), the disease based on the mapped one or more symptoms with the set of parameters associated with the target disease.

17. The method as claimed in claim 16, wherein the method comprises the step of
collecting, by the one or more processors (202), a plurality of points of data chosen by a plurality of medical experts to gather, refine and enrich the medical knowledgebase.

18. The method as claimed in claim 16, wherein the method further comprises the step of displaying by an interface unit, operatively coupled to the system configured to display a plurality of information associated with the diagnosed disease,
The method as claimed in claim 16, wherein the method further comprises the step of
performing, by the one or more processors (202), a feedback analysis executed by the plurality of medical experts, wherein the feedback analysis is based on a plurality of triages performed by the plurality of medical experts mimicking a plurality of clinical scenarios, wherein each said triage includes the target disease, age, gender, one or more symptoms and context applicable to the plurality of clinical scenarios.

19. The method as claimed in claim 16, wherein the method further comprises the step of generating, by the one or more processors (202), one or more derived symptoms based on one or more symptom questions and an initial set of complaints provided by a user.

20. The method as claimed in claim 16, wherein the one or more symptoms includes the set of initial symptoms and the one or more derived symptoms.

21. The method as claimed in claim 16, wherein the method further comprises the step of
finding out a plurality of abnormalities or medically incorrect responses generated by the system (110).

22. The method as claimed in claim 16, wherein the method further comprises the step of
improving and fixing, by the one or more processors (202), a knowledge graph comprising one or more knowledge features associated with the medical knowledgebase;
Updating, by the one or more processors (202), the one or more knowledge features in the knowledge graph;
Adding, by the one or more processors (202), a new feature to the knowledge graph.

23. The method as claimed in claim 22, wherein the knowledge graph is coupled with appropriate visual representations of a medical knowledge that is easily and accurately comprehensible to the user.

24. The method as claimed in claim 16, wherein the method further comprises the step of
Allowing, by the one or more processors (202), for factoring in a plurality of dimensions of medical knowledge whose dynamic nature works to augment the accuracy of diagnosis prediction.

25. The method as claimed in claim 16, wherein a user interface operatively coupled to the one or more processors (202) is configured with an intuitive dialogue between the user and the system (110).

26. The method as claimed in claim 25, wherein the user interface is configured with one or more clickable interactive humanoid representations, wherein the user chooses one or more clickable interactive humanoid representations that represent the part that the user has the pain at.
